(19) United States
(12) Patent Application Publication
(10) Pub. No.: US 2007/0259196 A1
(43) Pub. Date: Nov. 8, 2007

(54) ANTIMICROBIAL SHEET AND USE OF SAID SHEET

(75) Inventors: Thomas Ruhle, Weinheim (DE); Dirk Schubert, Hirschberg (DE); Jurgen Henke, Viernheim (DE); Achim Gruber, Schonau (DE); Judith Haller, Bruchsal (DE); Thomas Schindler, Zwingenber (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Carl Freudenberg KG, Weinheim (DE)

(21) Appl. No.: 11/619,486

(22) Filed: Jan. 3, 2007

(30) Foreign Application Priority Data
May 3, 2006 (DE) 10 2006 020 791.2

(51) Int. Cl. A61L 9/01

(52) U.S. Cl. 428/540

(57) ABSTRACT

A sheet consisting of a carrier medium, where at least one antimicrobial substance is incorporated into the carrier medium, is, in view of the task of specifying a sheet that exhibits high reactivity of the antimicrobial substance, characterized by the fact that the substance is in colloidal and/or nanoscale form. Moreover, the sheet is characterized by the fact that the substance is contained in a layer that is at least area wise interrupted or consists of unconnected partial layers. Moreover, a use of the sheet as a cleaning article or in a cleaning article is described.

### TECHNICAL FIELD

The invention concerns a sheet consisting of a carrier medium, where at least one antimicrobial substance is incorporated into the carrier medium. Moreover, the invention concerns the use of a sheet as a cleaning article or in a cleaning article.

### PRIOR ART

From the prior art there are known sheets and cleaning articles that consist of a carrier medium in which an antimicrobial substance is incorporated. In this case an antimicrobial substance has antibacterial, antiviral, or antimycotic activity and/or acts against spores.

The known sheets or cleaning articles, however, have considerable disadvantages with regard to the reactivity of the antibacterial and/or antimicrobial substance.

The known loading of sheets with antimicrobial substances hinders rapid availability of the antimicrobial substance, since its molecules are frequently not able to be mobilized sufficiently rapidly. This has to do with the fact that the molecules of the substance are present in a bulk phase and are shielded by surrounding molecules. This problem arises in particular when coatings exceed a critical thickness and surface spread.

### PRESENTATION OF INVENTION

The invention therefore is based on the task of providing a sheet of the kind indicated at the start that is characterized by high reactivity of the antimicrobial substance.

In accordance with the invention this task is solved with the characteristics of Claim 1. Accordingly the antimicrobial substance is in colloidal and/or nanoscale form.

In accordance with the invention, it was learned that a colloidal and/or nanoscale substance has especially high reactivity when it is brought into contact with bacteria, viruses, fungi or spores. In addition, it was known that the sheet, in accordance with the invention very readily releases the active substance to media that are in contact with the sheet. To that extent, the sheet in accordance with the invention is characterized by a high capacity for delivery of the antimicrobial substance.

Colloids are disperse systems in which substances are distributed in a dispersion agent so that their particles have a dimension of 10 to 1000 Angstroms in at least one spatial direction and consist of 10³ to 10⁹ atoms.

Nanoscale structures are understood to mean regions of any morphology that have dimensions in the nanometer range in at least one spatial direction.

Colloids are an intermediate state of heterogeneous and homogeneous mixtures. The ratio of surface area to volume is very high in the case of colloidal and nanoscale structures, due to which high reactivity is ensured. The colloidal distribution moreover enables problem-free diffusion or release of the particles of the substance from a carrier medium. To that extent, the antimicrobial substance can exhibit high mobility and can kill or neutralize bacteria, viruses and spores efficiently and rapidly.

The task indicated at the start is solved as follows. By suitable variations of the process parameters in the application of the substance, its morphology can be affected. In particular, by subsequent treatment of the deposited particles of the substance, the particle shape, particle size, layer thickness and degree of loading of the antimicrobial substance can be adjusted. To this extent, the timewise delivery profile and thus, the reactivity of the antimicrobial substance can be precisely adjusted.

The substance can be distributed in a carrier medium. The entire effective surface could be loaded with the substance. Through this, the antimicrobial substance is advantageously homogeneously distributed over the entire sheet. Because of this, the substance can be active over all of the surface area of the sheet.

The substance could be distributed in a layer applied to the carrier medium or integrated into such a layer. Here, it is conceivable for the substance to be colloidally distributed, both in the carrier medium and the carrier medium, moreover, to be provided with a coating that contains the substance in colloidal or noncolloidal state. Through this combination, an especially long lasting antimicrobial mode of activity of the sheet can be realized, since the substance from the carrier medium already becomes active before the coating wears away.

For example, a printing paste could function as the layer. The provision of a printing paste enables an especially cheap production process. Through this, process costs such as those that arise, for example, in the evaporation out of the active substance, are effectively avoided.

The layer could be interrupted at least areawise or could consist of unconnected partial areas. In connected areas, the substance cannot be sufficiently mobilized because of the closed surfaces and therefore it cannot freely develop its reactivity. Interruptions in the layer produce edge regions where the antimicrobial substance is clearly more reactive and thus, can be mobilized more rapidly. The formation of partial layers, moreover results in a large number of edges and fissures at which the described effect can develop.

Against this background, the task indicated at the start is additionally solved with a sheet that has the characteristics of Claim 5.

In order to avoid repetitions with respect to inventive activity, reference is made to the embodiments below.

The layer or the partial layers could be designed as island structures. The formation of island structures could be achieved by sputter deposition. In this process a substrate is brought into the vicinity of a target so that atoms knocked out of the target can condense on the substrate and form a layer. The atoms that are knocked out of the target are atoms of the antimicrobial substance. Island structures can be formed by this method. Here the islands form clusters or monoclusters. In their totality, these island structures exhibit a very large surface area with the formation of a large number of edges, at which molecules or atoms of the antimicrobial substance can be mobilized sufficiently rapidly, for example, to interact with bacteria and to neutralize or kill them.

Against this background, it is likewise conceivable to form the layer with a silver-containing printing paste. Here, the printing paste could consist of a silver dispersion. This specific embodiment enables a cheap application of patterns, letters or symbols, in order to give the user an indication of the technical use of the sheet. Letters, symbols or patterns often have to be applied in order to indicate their intended use. Through the use of the printing paste or silver dispersion, a separate coating of the sheet is not necessary, since the patterns or symbols themselves can function as an antimicrobial layer.

Moreover, it is conceivable for the application of the layer to take place by wet chemical means through impregnation with a silver precursor, and subsequent conversion of the precursor to metallic silver. Silver nitrate (AgNO₃), silver sulfate (AgSO₄)₂, organometallic complexes or metallocenes can function as silver precursors. The use of these silver precursors allows a locally selective creation of zones and regions in which the silver is present in metallic form, optionally through the use of masks or templates. The silver precursor remains in the other regions, possibly after removal of a mask or template.

Moreover, it is conceivable to impregnate a sheet with a silver dispersion or cleaning solution that consists of a silver dispersion. The impregnated sheet can then be used in dry form or in wetted state, for example, as a disposable cleaning article. Impregnation is a cheap and rapid method for applying silver to a sheet.

The layer or the partial layers could have a thickness of 0.05 to 1000 nm. This range of layer thicknesses proved to be particularly favorable for achieving sufficient mobility of the atoms or molecules of an antibacterial and/or antimicrobial and antimycotic substance.

Against this background, the layer or partial layers could have an areal density of 5-1000 mg/m². This loading is quite sufficient for a few uses of the sheet, so that it can be used as a single-use article or disposable article. Moreover, it is conceivable to provide for a substance load up to 10,000 ppm (mg/kg) on a carrier medium.

The carrier medium could consist of fibers. In this way, the carrier medium makes available a fissured surface for the application of antimicrobial substances. Thus, layers of the active substance that are deposited on the carrier medium are subjected to fissuring.

Against this background, the active substance could be incorporated into a number of different types of fiber, where other types of fibers would have a clearly lower load of the active substance. The fissuring effect is increased even more by this. Really quite specifically, the nanoscale and/or colloidal structures of the substance could be preferably deposited on hydrophobic, especially polyolefinic, fibers of a nonwoven material, where hydrophilic, especially viscose fibers, could largely be free of the active substance. In this way, it is possible to deposit the active substance selectively onto a particular fiber type in a nonwoven material that consists of a fiber mixture. The fissuring of the substance layer or the substance load then arises through the fiber structure of the carrier medium.

The carrier medium could consist of a nonwoven material. The use of a nonwoven material allows the porosity to be specified by the appropriate choice of the fiber density or fiber thickness. Here it is conceivable that the nonwoven material is even formed of nanofibers, which ensure a very small pore size. Nanofibers usually have a diameter that is less than 1 μm and preferably is between 50 and 500 nm. Through the use of nanofibers the fissuring effect described above can be increased even further. Moreover, nonwovens are characterized by high absorption capacity and therefore can function as cleaning towels, which absorb liquids. In particular, such cleaning towels could consist of multilayer nonwovens, where each sheet could have a different pore size, a different fiber material or a different average fiber diameter. Quite specifically, at least one sheet could consist of split fibers. This fiber type can be easily split and/or consolidated by water jet treatment. At least one sheet could consist of staple fibers or endless fibers. These fiber types can readily be consolidated and/or entangled by water jet treatment.

The carrier medium could consist of polymers. Against this background it is especially conceivable that thermosetting plastics like polypropylene, polyethylene, or polyester as well as polyamide can be used. These materials are especially suitable for preparation of fiber-containing nonwovens, since the fibers consisting of these polymers can be melted together under the effect of heat and thus firmly consolidated. This enables the consolidation of fiber knitted materials.

Polypropylene and polyethylene are especially suitable for deposition of silver. Silver can readily be deposited on polypropylene or polyethylene and forms a solid bond with these materials. Silver can readily be deposited on these materials in particular by sputtering. A solid bond results from van der Waals forces or a chemical bond. By thermal or plasma treatment of polypropylene and/or polyethylene, their surfaces become activated and the adhesion of silver to the surfaces is improved.

The carrier medium could be designed as a multiuse latex glove. This design advantageously allows the use of products that already exist and allows them to be provided with antimicrobial substances cheaply.

The carrier medium could consist of chitosans and/or cyclodextrins. These materials proved to be especially suitable for incorporation of colloidal silver and other substances like fragrances.

Against this background, fragrances could be incorporated into the carrier medium. Through this bad odors can be neutralized, adsorbed or suppressed or masked.

The chitosans and/or cyclodextrins can be loaded with silver and/or fragrances and fixed onto the actual carrier medium. The chitosans and/or cyclodextrins enable controlled and long-lasting delivery of incorporated colloidal silver or incorporated fragrances.

Metallic silver, which is not in ionic form, could be deposited in layer silicates or zeolites. Here, it is advantageous that the silver can be in the form of nanoclusters in channel structures in the layers or zeolites. First, silver oxide forms at the edge of the channel structure and then diffuses out and is converted to ionic silver. Then, the next layer of a nanocluster is converted to silver oxide, and the process repeats. In this way, a depot effect can be achieved, in which ionic silver can be released in a defined and controlled way over a long period of time. Against this background, the following method is conceivable. First a silver salt solution, especially an aqueous silver nitrate solution, is produced. The zeolite or the carrier medium is immersed in the solution. Then this is followed by a two-step thermal treatment. First the silver salt is converted to silver oxide in an air atmosphere. This silver oxide is reduced to metallic silver in a hydrogen atmosphere. In this way metallic silver is present within the pores of the layer silicate. An especially homogeneous and uniform distribution of metallic silver within very fine channel structures is possible through this specific method. Clusters of metallic silver of different sizes can result, in dependence on the pore size or on the channel diameter. To this extent, a polymodal distribution of nanoscale silver structures is possible within a layer silicate.

Against this background, it is conceivable that, in addition to fragrance molecules, colloidal silver is incorporated into channel structures and call be released and develop its antimicrobial effect in a manner analogous to that of the fragrances.

It is also conceivable for colloidal silver to be incorporated into the cyclodextrins and chitosans instead of fragrance molecules. In this embodiment, it is possible for silver to exit and, in counterflow, odors to be trapped by the resulting voids in the cyclodextrins and chitosans. This design results in a savings of costly fragrances.

The antimicrobial substance could consist of silver. Silver is especially suitable as an antimicrobial substance, since it is nearly nontoxic for humans. Moreover, silver has a relatively low allergenic potential. In low concentrations, silver acts as an antiseptic substance on a large number of infectious microbes over a long period of time. Moreover, most of the known bacteria do not have any resistance to silver.

The antimicrobial substance could consist of at least one side group element. Side group elements are characterized by antimicrobial activity. Against this background, it is conceivable for a number of side group elements to be jointly present in the layers and/or the carrier material in order to counteract different species of bacteria selectively. It was shown in experiments that the antimicrobial substances can be ranked with reference to antimicrobial efficacy. This can be shown as follows: Silver is the most effective substance, followed by mercury, copper, cadmium, chromium, lead, cobalt, gold, zinc, iron, and finally manganese. Against this background, it is also conceivable to use main group elements that have an antimicrobial effect.

The antimicrobial substance could comprise a gold-silver mixture or could consist exclusively of gold-silver mixture. Mixtures of this kind show particularly high antimicrobial efficacy. Surprisingly, it turned out that the presence of gold increases the antimicrobial effect of silver even further. Against this background, it is conceivable to dope silver with gold. It is also conceivable to form islands or clusters that consist either of only gold or only silver or that consists of mixtures of these substances. Islands or clusters of different composition could be present next to each other.

Aluminum could be mixed into the substance. Aluminum causes a brightening or improved visual appearance of the coating over the long term, since silver, for example, turns brown in oxidation processes. This leads to an unpleasant appearance of the coating or of the overall sheet. Aluminum, moreover, causes a modification of the delivery rate of the antimicrobial substance.

The substance could be part of a supported system. This means a system in which the actual antimicrobial substance is integrated into carrier substrates. The carrier substrates could consist of carbon blacks or oxides. By the addition of particles of antimicrobial substance to carrier substrates, it is ensured that the individual particles of the antimicrobial substance do not agglomerate. The activity of the active substance is clearly improved through this. The carrier substrates themselves could be integrated into the actual carrier medium.

The sheet could be provided with a plasma coating. The delivery rate of the antimicrobial substance can be adjusted via a plasma coating. To this extent, the microbial effect of the sheet can be adjusted. A plasma coating is a production process in which materials are coated with thin layers that are extracted from a plasma under the effect of an electrical voltage. A workpiece to be treated is, after very thorough cleaning, put into a vacuum chamber and secured there. The chamber is evacuated until a residual gas pressure in the high vacuum or ultrahigh vacuum range is achieved, in each case according to the process. Then a working gas, most often argon, is admitted via valves and a low pressure plasma is initiated by various methods for delivering energy, for example, microwaves, high frequency, electrical discharge.

The task indicated at the start, moreover, is solved with the characteristics of Claim 18.

The use of a sheet described herein as a cleaning article or in the cleaning article is especially advantageous, since the antimicrobial substance has high reactivity and the sheet shows excellent substance delivery behavior. When using the sheet in accordance with the invention as is or in a cleaning article therefore, it is, in particular, ensured that the antimicrobial substance will remain on a cleaned surface. A long lasting and persistent disinfectant and cleaning effect is achieved through this.

Against this background, it is conceivable that the cleaning article is designed as a cleaning towel, especially as a single-use cleaning towel. Because of the extremely low loading with antimicrobial substance, its effect can already be used up after one or a few uses of the cleaning towel. The design of a cleaning towel as a single-use cleaning towel is especially inexpensive, since the antimicrobial substances, which for the most part are very expensive, can be applied in an extremely finely dosed way. Single-use cleaning towels have an advantage over multiple-use cleaning towels, since they cannot form colonies of contamination after a single use, since they are immediately disposed of.

Multiple-use towels are more expensive than single-use towels, since they contain more antimicrobial substance. Still, multiple-use towels cannot be used for a longer time in proportion to the amount of the antimicrobial substance, since they often cannot be used in accordance with their function after only a relatively few cycles of use, because of contamination.

The sheet could be used in floor cleaning and/or floor disinfection. Against this background it is conceivable that the sheet will find use in a wiping mop. A wiping mop most often consists of a number of textile strips that serve to absorb liquid. These strips could be formed by the sheets in accordance with the invention. This specific design enables the use of the antimicrobial substance in hospitals, nursing homes and other sites, for example, large kitchens, in which it is undesirable for bacteria to form on the floor.

Moreover, it is conceivable that the sheet can be designed as a nonwoven material, a woven material, knitted material, or as a yarn. The use of a nonwoven material is advantageous with regard to the ability to establish the porosity. Woven fabrics and knitted fabrics are characterized by high mechanical stability and can have different fiber types in a blend. The use of different fiber types, specifically fibers of different materials, enables the selective addition of the active agent onto the fibers. Yarns are advantageous when the sheet is used in wiping mops, especially loop mops. The yarn in this case replaced the strips described above.

Moreover, it is conceivable that the sheet is designed as a film. It is especially conceivable that the sheet is designed as a freshness keeping film or packaging film for foods. The coating of the sheet with antimicrobial substances effectively enables the suppression of the formation of bacteria, which can spoil foods.

Moreover, it is conceivable that the carrier medium is designed as a foam body. When using an open-cell foam, it is even conceivable that the interior of the foam is impregnated with the antimicrobial substance.

The foam body could be used as a cleaning sponge. Through this it is ensured that liquid absorbed by a foam body becomes disinfected or that growth of bacteria in it is inhibited. This is advantageous when the foam body is used as a sponge in areas where food is present such as bars or tables in restaurants.

In each case according to use, hygienic conditions can be easily improved, i.e., bacterial growth can be suppressed or, in the extreme case, bacterial eradication can be achieved.

The sheets described here can be used in nearly all hygienic or cosmetic products, because of their antimicrobial activity. Here baby wipes, diapers, body care towels, face towels or products for incontinent patients are especially conceivable.

Also, formation of a biofilm in water treatment can also be suppressed with these materials. It is also conceivable to use the sheets described here as filters in air conditioners or aeration systems. In this way, harmful pathogens and microbes in the air that is breathed can be effectively reduced or even removed.

When the advantageous antimicrobial properties of silver are combined with the properties of nanoscale systems, novel material properties result, which are essentially due to a high surface/volume ratio.

The microbially active silver ions arise as silver oxide on the nanoparticle surface through the effect of atmospheric oxygen and moisture from the environment. The oxide layer itself has an essentially constant thickness independent of the particle size. This means that the microbially active volume of the total volume increases significantly with decreasing particle size.

If silver is present in nanoscale form, significant advantages result. Due to the more finely divided presentation, very much less is required than would be required if coarse-particulate silver were used. A clearly larger amount of the silver becomes accessible to the environment because of the larger surface/volume ratio. In this way, the ionic silver can be mobilized clearly more rapidly. A true depot effect ensuring long lasting activity is obtained.

Materials that would otherwise not be accessible to a silver finish can be provided with nanoscale silver. Thus, for instance, coarse silver particles could not be spun into all polymer fibers, for example, since the nozzles become plugged.

If one wishes to use nanoscale silver in thermosetting plastics and elastomers, in principle, there are two possibilities for application:

Application of nanoscale silver to a substrate surface, namely by chemical means or by evaporation. Since the silver sits on the surface in this case, it can act very rapidly. In addition, the silver delivery profile can be established very readily through the morphological organization (form, size) of the silver nanostructures.

In compounding nanoscale silver, nanoscale silver can be compounded simultaneously with other fillers. In each case according to the hydrophilicity of the polymer, concentrations of 500-2000 ppm silver may be necessary for a sufficient effect here. However, a portion of the silver within the volume is not accessible in this case. Moreover, the effect is delayed, since the silver first must diffuse to the polymer surface.

There are now various possibilities for advantageously organizing and developing the teaching of this invention further. For this one should refer on the one hand to the dependent claims, and on the other hand, to the following illustration of preferred embodiment examples of the teaching in accordance with the invention by means of the tables.

In connection with the illustration of the preferred embodiment, examples by mew-s of the tables, preferred organizations and developments of the teaching are also generally illustrated.

### DESCRIPTION OF TABLES

In the tables
Table 1 shows the results of microbiological tests in connection with bacteria of type Escherichia coli,
Table 2 shows the results of microbiological tests in connection with spores of type Aspergillus niger,
Table 3 shows the olfactory evaluation of silver-loaded samples, and
Table 4 shows the eradication rate of bacteria on glass panes.

### EMBODIMENTS OF THE INVENTION

### EMBODIMENT EXAMPLES

The sheets of two test series 1 and 2 described below were prepared and tested as follows:

### Experimental Series 1:

Various silver loads were applied by a magnetron sputtering process to sheets, which consisted of carrier media of a nonwoven material that has a areal weight between 50 and 500 mg/m².

The nonwoven material consists of polymer fibers. Additionally, it contains natural fibers, namely cellulose fibers. Specifically, nonwovens that contain viscose, polypropylene and polyethylene terephthalate fibers in a blend were used.

Sample 2 has a silver load of 10.5 mg/m². Samples 3 to 6 have silver loads of 29.4, 56.7, 115.5 and 231 mg/m², respectively. Sample 1 does not have a silver load and is a control sample.

As the antimicrobially active substance, the carrier material contains silver, which is in colloidal and/or nanoscale form. This is brought about by the generation of essentially rectangular nanoparticle island structures of silver with an edge length in the range of 5 nm.

The island structures that result on the carrier material have a specific surface that is greater than the surface of a closed nanolayer with a thickness of 5 nm.

This is why the delivery rate of a carrier medium that has rectangular island structures with edge length 5 nm is clearly greater than that of a completely coated carrier medium.

The rectangular island structures were detected by SIMS. Specifically, it was found that the nanoscale and/or colloidal silver structures are preferably deposited on the polyolefin fibers of the nonwoven that is used. The viscose fibers are largely free of silver. In this way it is possible to deposit silver selectively on a particular fiber type in a nonwoven that consists of a fiber blend.

By appropriate variation of the process parameters and the load, the size of the islands and the range of their size distribution can be controlled. In this way the specific surface and thus the delivery profile of the antimicrobially active silver can be adjusted. Specifically, polymodally distributed nanostructures can be generated by controlled adjustment of the process parameters. These structures will each have a variously high number of unsaturated surface atoms. Through this they have a variously high reactivity or microbiological activity.

Samples 1 to 6 were subjected to a test for antimicrobial finish according to the generally known AATCC Method 100, which is used for textile materials.

The results of this test are shown in Table 1. Table 1 shows the eradication rate of Escherichia coli cells as a function of the silver load. In Table 1, the silver load is given in mg/m² in the first column. The second column shows the microbial count in units CFU/mL (colony-forming units/mL) after 24 h, and the third column shows the eradication rate after 24 h in percent. The fourth and fifth columns are similarly organized.

Table 2 shows the results of a microbiological test conducted with spores of type Aspergillus niger on Samples 1 to 6. The samples 1 to 6 functioned as patterns for dishtowels (dishtowel master).

Because of its dark spores, Aspergillus niger is also called black mold. Aspergillus niger is very common food spoilage agent and destroyer of materials. It occurs in soil world wide. This mold fungus can destroy paper and packaging materials as well as leather and paints, even plastics and optical glasses. Diseases caused by Aspergillus niger include, in addition to allergic reactions, infections of the outer ear, pulmonary aspergillosis, inflammations of the peritoneum, inflammations of the endocardium, diseases of the nails as well as skin infections

The first column in Table 2 shows the silver load in mg/m². Quantity B in the second column qualitatively indicates if the relevant sample has been overgrown with spores after two days. The third column analogously indicates if the sample is overgrown after four days. (B) expresses, only qualitatively, that the growth is somewhat weaker. The (−) qualitatively indicates that there is no growth present.

Samples 1 to 6 in Test Series 1 was additionally subjected to an odor test.

For this, the samples set up as towels were stored for 48 h at 32° C. in 100 mL 10% milk solutions. Then the samples were removed and dried. The milk solutions and the samples, rewetted with 100 μL water after they have been dried, were olfactometrically evaluated. The samples were subjected to a blind test by 10 testers. The testers were requested to evaluate the solution or the samples on a scale based on the following qualitative evaluations:
Grade 6 intolerable,
Grade 5 highly irritating,
Grade 4 irritating,
Grade 3 clearly perceptible, but not yet irritating,
Grade 2 perceptible, not irritating,
Grade 1 not perceptible.

Table 3 shows the results of the evaluation.

Moreover, the rapid mobilizability of the silver ions was confirmed in rinse-out experiments.

Samples 1 to 6, 2.5×5 cm in size, were each stored in 100 mL water with pH values 3, 7 and 11, and the silver concentrations were determined. It was found that the delivery rate is the greatest in the first hour of storage. The antibacterial activity therefore is effective very rapidly, so that complete eradication of bacteria can be achieved after only 24 h. Nevertheless, after the end of the first hour, a moderate release rate is observed, which also guarantees a medium to long term effect.

In another test a reference sample with a silver load of 55 mg/m² was subjected to two complete standard wash cycles in a commercial washing machine with a commercial washing powder. After the first wash cycle, about 30% of the silver was still present on the towel. After the second wash cycle, eradication rates of up to 91.17% for bacteria of type Escherichia coli and 99.33% for bacteria of type Staphylococcus aureus could be detected on the towel.

### Experimental Series 2

Table 4 shows the results of a test in which glass panes were treated with different samples.

Samples of type A were used for this: Sheets with a carrier medium of nonwoven [material] or impregnated with a nanosilver dispersion. These sheets served as wiping towels for disinfection of glass panes In these sheets, the silver is colloidally dispersed on the carrier medium. The silver is homogenously distributed in the carrier medium.

To prepare these sheets, first a standard floor cleaner was provided with a silver concentration of 500 ppm silver.

This standard floor cleaner was applied to 10×10 cm samples, which were put into a beaker and left covered for about 17 h overnight at room temperature. After 17 h, each sample was cut in half. One half was directly pressed out gently between two hand towels, while the other half was gently rinsed for about 30 seconds with tap water and then gently pressed out.

All the samples of type A were then dried for 3 h at 100° C. in a circulating air oven.

### Use of Sample of Type B:

Moreover, glass panes were wiped with samples of type B (towel) that had been wetted with 120 mg/m² silver.

Class panes were specifically and reproducibly wiped a number of times with samples of type A and B. Then microbiological tests were carried out with the samples and the eradication rates were determined. This was done as follows:

2.5×5 cm samples of types A and B were stamped out and loaded with 20 mL water. The samples were moved back and forth with 50 N normal force on 20 cm long and 5 cm wide glass panes in 50 oscillation cycles.

For bacteria of type Escherichia coli an eradication rate of 95.7% was obtained when using the wetted samples (towels). An eradication rate of 99.83% resulted when using samples that had been impregnated with the standard floor cleaner with a silver concentration of 500 ppm.

An eradication of >99.89% was obtained for bacteria of type Staphylococcus aureus when using the wetted samples. An eradication of 99.93% was seen when using the impregnated samples.

Glass panes that were not wiped did not show any eradication, i.e., on these glass panes the bacteria were present in the initial concentrations.

Finally, it should expressly be pointed out that said embodiment examples merely serve for discussion of the claimed teaching, but do not limit it to these embodiment examples.

TABLE 1

| | Escherichia coli | | | |
|---|---|---|---|---|
| | Zellzahl 0 h: | | Zellzahl 0 h: | |
| | 1.2 × 10⁶ Zellen pro | | 3.6 × 10⁷ Zellen pro | |
| Silber- | Musterprobe \textcircled{2} | | Musterprobe \textcircled{3} | |
| Beladung | | Abtötungsrate | KBE/ml | Abtötungsrate |
| [mg/m²] | KBE/ml | nach 24 h | nach 24 h | nach 24 h |
| \textcircled{1} | nach 24 h \textcircled{4} | [%] \textcircled{5} | \textcircled{4} | [%] \textcircled{5} |
| 0 | 1.3 × 10⁷ | 0% | 2.1 × 10⁸ | 0% |
| 10.5 | <100 | >99.9999% | 1.2 × 10⁸ | 43% |
| 29.4 | <100 | >99.9999% | <100 | >99.9999% |
| 56.7 | <100 | >99.9999% | 5.7 × 10⁴ | 99.9952% |
| 115.5 | <100 | >99.9999% | <100 | >99.9999% |
| 231 | <100 | >99.9999% | <100 | >99.9999% |

Key:
\textcircled{1} Silver load (mg/m²)
\textcircled{2} Cell count, 0 h: 1.2 × 10⁶ cells per master sample
\textcircled{3} Cell count, 0 h: 3.6 × 10⁷ cells per master sample
\textcircled{4} CFU/mL, after 24 h
\textcircled{5} Eradication rate after 24 h (%)

TABLE 2

| | Aspergillus niger | | | |
|---|---|---|---|---|
| Silber- | 9 × 10⁸ Sporen pro | | 8 × 10⁸ Sporen pro | |
| Beladung | Spültuch-Muster \textcircled{2} | | Spültuch-Muster \textcircled{3} | |
| [mg/m²] \textcircled{1} | 2 Tage \textcircled{4} | 4 Tage \textcircled{5} | 2 Tage \textcircled{4} | 4 Tage \textcircled{5} |
| 0 | B | B | B | B |
| 10.5 | B | B | B | B |
| 29.4 | — | B | — | B |
| 56.7 | — | B | — | B |
| 115.5 | — | (B) | — | B |
| 231 | — | (B) | — | B |

B growth
(B) growth somewhat weaker
— no growth
Key:
\textcircled{1} Silver load (mg/m²)
\textcircled{2} 9 × 10⁵ spores per dish towel master
\textcircled{3} 9 × 10⁶ spores per dish towel master
\textcircled{4} 2 days
\textcircled{5} 4 days

TABLE 3

| Silberbeladung | Bewertung | Bewertung der getrockneten |
|---|---|---|
| [mg/m²] \textcircled{1} | der Milchlösung \textcircled{2} | Tücher + 100 μl H₂O \textcircled{3} |
| 0 | 4.7 +/− 0.41833 | 5.7 +/− 0.28868 |
| 10.5 | 4.5 +/− 0.77055 | 5.2 +/− 0.14434 |
| 29.4 | 3.8 +/− 0.33541 | 5.0 +/− 0.25 |
| 56.7 | 3.3 +/− 0.41833 | 4.7 +/− 0.28868 |
| 115.5 | 3.4 +/− 0.1118 | 4.7 +/− 0.28868 |
| 231 | 3.7 +/− 0.22361 | 4.3 +/− 0.28868 |

Key:
\textcircled{1} Silver load (mg/m²)
\textcircled{2} Evaluation of milk solution
\textcircled{3} Evaluation of dried towels + 100 μL H₂O

TABLE 4

| | Keim \textcircled{2} | |
|---|---|---|
| | Escherichia | Staphylococcus |
| | coli | aureus |
| | Abtötungsrate | Abtötungsrate |
| Variante \textcircled{1} | [%] \textcircled{3} | [%] \textcircled{3} |
| Unbehandelte Scheibe | 0 | 0 |
| (Referenz) \textcircled{4} | | |
| Bedampftes Tuch (120 mg/m²) \textcircled{5} | 95.7 | >99.89 |
| Variante mit Lotion (500 ppm | 99.83 | 99.93 |
| Ag) \textcircled{6} | | |

Key:
\textcircled{1} Variation
\textcircled{2} Microbe
\textcircled{3} Eradication rate (%)
\textcircled{4} Untreated pane (reference)
\textcircled{5} Wetted towel (120 mg/m²)
\textcircled{6} Variation with lotion (500 ppm Ag)

1. A sheet comprising a carrier medium, at least one antimicrobial substance is incorporated into the carrier material, wherein the substance is in colloidal and/or nanoscale form.

2. A sheet as in claim 1 wherein the substance is distributed in the carrier medium.

3. A sheet as in claim 1 wherein the substance is incorporated into a layer applied to the carrier medium.

4. A sheet as in claim 3 wherein the layer is interrupted at least areawise or consists of nonconnecting partial layers.

5. A sheet comprising of a carrier medium, at least one antimicrobial substance is incorporated into the carrier medium, the substance is contained in a layer that is at least areawise interrupted or consists of unconnected partial layers.

6. A sheet as in claim 4 wherein the layer or the partial layers are formed as island structures.

7. A sheet as in claim 5 wherein the layer or partial layers have a thickness of 0.05 to 1000 nm.

8. A sheet as in claim 5 wherein the layer or partial layers have an areal density of 5 to 1000 mg/m².

9. A sheet as in claim 1 wherein the carrier medium consists of fibers.

10. A sheet as in claim 9 wherein the active substance is integrated into a number of different types of fiber, where other types of fiber have a clearly lower load or no load at all of the active substance.

11. A sheet as in claim 1 wherein the carrier medium consists of a nonwoven material.

12. A sheet as in claim 1 wherein chitosans and/or cyclodextrins are incorporated into the carrier medium.

13. A sheet as in claim 1 wherein fragrances are incorporated into the carrier medium.

14. A sheet as in claim 1 wherein the antimicrobial substance consists of silver.

15. A sheet as in claim 1 wherein the substance consists of at least one side group element.

16. A sheet as in claim 1 wherein the antimicrobial substance consists of gold or a silver-gold mixture.

17. A sheet as in claim 1 wherein aluminum is mixed into the substance.

18. The use of a sheet as in claim 1 as a cleaning article or in a cleaning article.
